(19) United States
(12) Patent Application Publication
(10) Pub. No.: US 2020/0046781 A1
(43) Pub. Date: Feb. 13, 2020

(54) BACTERIAL AMYLOID INDUCED PROTEINOPATHIES AND TREATMENTS THEREFOR

(71) Applicant: UNIVERSITY OF LOUISVILLE RESEARCH FOUNDATION, INC., Louisville, KY (US)

(72) Inventor: Robert FRIEDLAND, Louisville, KY (US)

(21) Appl. No.: 16/339,741

(22) PCT Filed: Oct. 5, 2017
(86) PCT No.: PCT/US17/55394

§ 371 Date: Apr. 5, 2019

Related U.S. Application Data
(60) Provisional application No. 62/404,447, filed on Oct. 5, 2016.

(51) Int. Cl. A61K 35/74; A61P 25/14; A61P 25/28

(52) U.S. Cl. CPC A61K 35/74; A61P 25/28; A61P 25/14

(57) ABSTRACT

Methods of preventing, inhibiting and/or decreasing protein deposits of a human amyloid protein and/or neuroinflammation in the central nervous system by reducing an amount of exposure to a pathogenic bacterial amyloid in the gastrointestinal tract are disclosed. Also disclosed are methods of preventing, inhibiting and/or treating a proteinopathy by reducing an amount of exposure to a pathogenic bacterial amyloid in the gastrointestinal tract.

### RELATED APPLICATION(S)

This application claims the benefit of priority to U.S. Provisional Application Ser. No. 62/404,447, filed Oct. 5, 2016, the contents of which are hereby incorporated by reference in its entirety.

### FIELD OF THE INVENTION

The present disclosure relates generally to the field of neurodegenerative disorders, and particularly to the diagnosis and treatment of proteinopathies, including Parkinson's Disease, Alzheimer's Disease, amyotrophic lateral sclerosis and related disorders.

### BACKGROUND

It is now believed that Parkinson's Disease (PD), Alzheimer's Disease (AD), amyotrophic lateral sclerosis (ALS), Lewy body dementia, fronto-temporal lobar degeneration, cortical basal ganglionic degeneration, multiple system atrophy, progressive supranuclear palsy, and related conditions involve the formation of transmissible self-propagating prion-like proteins. However, the initiating factors responsible for the creation of these misfolded nucleating factors are unknown. In each case, neurodegeneration is preceded by the development of pathogenic aggregations of amyloid proteins (e.g., alpha-synuclein (AS), amyloid beta (AB), Tau, FUS, TDP43) in the central nervous system and, therefore, these conditions are known as proteinopathies.

Amyloid patterns of protein folding are highly conserved through evolution and are widely distributed in the world, and similarities of tertiary protein structure between human and microbial amyloid proteins may be involved in the creation of prion-like agents through molecular mimicry.

The largest opportunity for exposure to foreign proteins is found in the human gastrointestinal tract, which houses the human microbiome or microbiota (including viruses, bacteria and other agents). The microbiome of the human gastrointestinal tract includes a complex ecosystem of approximately 300 to 500 bacterial species, comprising nearly 2 million genes, and it has been estimated that the number of bacteria within the gut is approximately 10 times that of all of the cells in the human body. The microbiome is comprised mainly of anaerobic bacteria from the genera Bacteroides, Porphyromonas, Bifidobacterium, Lactobacillus, and Clostridium, with anaerobic bacteria outnumbering aerobic bacteria by a factor of 100 to 1000:1. See, e.g., Quigley (2013), Gastroenterol. Hepatol (NY) 9(9):560-569.

### SUMMARY

The present invention depends, in part, upon the demonstration that bacterial amyloid proteins present in the gastrointestinal tract are capable of inducing the first steps in the development of amyloidogenic neurodegenerative disease. This has been demonstrated by controlled experiments in an animal model in which amyloid deposits in neurons in the gut and brain were measured and compared. These experiments, described below, demonstrated that animals receiving oral administration of bacteria producing a bacterial amyloid protein exhibited greater amyloid deposits in the gut and brain neurons than animals receiving oral administration of control bacteria incapable of producing the bacterial amyloid. Additional experiments in the roundworm Caenorhabditis elegans support these findings. This is believed to be the first demonstration of causation of amyloidogenic neurodegenerative disease symptoms by bacterial amyloid proteins produced by bacteria in the gastrointestinal microbiome.

Thus, in one aspect, the invention provides methods of preventing, inhibiting and/or decreasing protein deposits of a human amyloid protein and/or neuroinflammation in the central nervous system by reducing an amount of exposure to a pathogenic bacterial amyloid in the gastrointestinal tract.

In another aspect, the invention provides methods of preventing, inhibiting and/or treating a proteinopathy by reducing an amount of exposure to a pathogenic bacterial amyloid in the gastrointestinal tract.

In some embodiments, the human amyloid protein is alpha-synuclein (AS), amyloid beta (AB), Tau, FUS or TDP43.

In some embodiments, the pathogenic bacterial amyloid is an amyloid produced by a bacterium of a phylum selected from the group consisting of Bacteroidetes, Proteobacteria, Firmicutes and Thermodesulfobacteria. In some of these embodiments, the pathogenic bacterial amyloid is the E. coli curli protein (or the E. coli A protein subunit) or a Bacteroidetes, Proteobacteria, Firmicutes or Thermodesulfobacteria homologue of the E. coli curli protein or the E. coli A protein subunit.

In some embodiments, the reduction in exposure is accomplished by administering at least one antibiotic to reduce the relative abundance of the bacterial strain producing the pathogenic bacterial amyloid.

In some embodiments, the reduction in exposure is accomplished by administering at least one prebiotic to increase the relative abundance of non-pathogenic bacterial strains which will at least partially displace the bacterial strain producing the pathogenic bacterial amyloid.

In some embodiments, the reduction in exposure is accomplished by administering at least one prebiotic to reduce the relative abundance of the bacterial strain producing the pathogenic bacterial amyloid relative to a non-pathogenic bacterial strain which will at least partially displace the pathogenic strain.

In some embodiments, the reduction in exposure is accomplished by administering probiotics to provide non-pathogenic bacterial strains which will at least partially displace the bacterial strain producing the pathogenic bacterial amyloid.

In some embodiments, the reduction in exposure is accomplished by administering a pharmaceutical preparation with an active agent that alters the pathogenic bacterial amyloid to a nonpathogenic form (e.g., a disaggregated or other form incapable of cross-seeding a human amyloidogenic protein).

In some embodiments, the reduction in exposure is accomplished by administering a pharmaceutical preparation with an active agent that alters the interaction between the pathogenic bacterial amyloid and a human amyloidogenic protein such that nucleation of the aggregated form is inhibited, prevented and/or reversed.

In some embodiments, the reduction in exposure is accomplished by administering a pharmaceutical preparation with an active agent that alters the interaction between the pathogenic bacterial amyloid and cells of the human gastrointestinal tract such that nucleation of the aggregated form of a human amyloidogenic protein is inhibited, prevented and/or reversed. In some specific embodiments, the interaction is uptake of the pathogenic bacterial amyloid into cells of the human gastrointestinal tract.

In some embodiments, the reduction in exposure is accomplished by administering a pharmaceutical preparation with an active agent which immunizes a subject against the pathogenic bacterial amyloid such that the subject's immune system reduces the relative abundance of the pathogenic bacteria.

These and other aspects and embodiments of the invention are illustrated and described below. Other compositions, methods and features will be apparent to one with skill in the art upon examination of the following drawings and detailed description. It is intended that all such additional compositions and methods and features are within the scope of the present invention.

### BRIEF DESCRIPTION OF THE FIGURES

The following drawings are illustrative of embodiments of the invention and are not meant to limit the scope of the invention as encompassed by the claims.

FIG. 1 shows rats exposed to bacteria expressing curli have enhanced AS deposition in hippocampus, striatum and gut neurons and increased growth of microglia. (a) alpha-synuclein (Syn-1) immunohistochemical staining in gut, striatum and hippocampus. Gut neurons containing AS deposits are indicated with arrows, and hippocampal neurons containing AS are indicated with arrowheads. Data are for animals exposed to mutant bacteria unable to produce curli (Mut Curli E. coli), curli-producing wild type bacteria (Curli E. coli) and Control (exposed only to vehicle). For the striatum and hippocampus images the bars are 20 μm For the bowel images the upper bar for the upper row of three bowel images is 250 μm and the lower bar for the lower row of three bowel images is 150 μm (higher magnification). Right panels represent quantification of the staining. *p<0.05 when compared to control. (b) Iba1 (allograft inflammatory factor) staining of neocortex, hippocampus and striatum. The upper bar is 250 μm and the lower bars are 20 μm The top images are lower magnification and the bottom three panels are higher magnification. *p<0.05 when compared to control.

FIG. 2 shows rats exposed to bacteria expressing curli have increased astroglial growth, enhanced deposition of aggregated AS, and increased expression in brain of IL6, TLR2 and TNF. Immunohistochemical staining in neocortex, hippocampus, striatum and substantia nigra. Right panels represent quantification of the staining. Data are for animals exposed to bacteria unable to produce curli (Mut Curli E. coli), curli producing wild type bacteria (Curli E. coli) and Control (exposed only to vehicle). Right panels represent quantification of the staining. (A) Glial fibrillary acidic protein (GFAP) staining of neocortex, hippocampus (CA3) striatum and substantia nigra. *p<0.05. (B) alpha-synuclein (α-syn) staining, without proteinase K treatment. *p<0.05. (C) alpha-synuclein (α-syn) staining following proteinase K treatment (PK+). Arrows represent deposits of proteinase K resistant AS in neurons. *p<0.05. (D) Interleukin 6 (IL6) staining in neocortex, hippocampus (CA3) striatum and Substantia nigra. *p<0.05. (E) Interleukin-1 (IL1) staining in neocortex, hippocampus (CA3) striatum and Substantia nigra. (F) Toll like receptor 2 (TLR2) staining in neocortex, hippocampus (CA3) striatum and Substantia nigra. *p<0.05. (G) Tissue necrosis factor (TNF) staining in neocortex, hippocampus (CA3) striatum and Substantia nigra. *p<0.05. For images (A-G) the bars are 20 μm.

FIG. 3 shows C. elegans exposed to E. coli expressing curli has enhanced AS aggregation. (a) Fluorescence microscopy of the anterior region of C. elegans expressing AS-YFP in the body wall muscle. Transgenic nematodes (Punc-54::AS::YFP) were age-synchronized and were fed for three days on lawns of either curli producing E. coli or its mutant strain unable to express curli (non-curli mutant). Fluorescence microscopy was performed on immobilized live nematodes and imaging was acquired under identical conditions for the nematode groups fed with different bacterial strains. C. elegans exposed to curli expressing bacteria (Curli) contained increased numbers of larger and brighter foci of AS-YFP as compared to those exposed to non-curli expressing mutant bacteria (Mutant). Scale bar=100 μm (b) Quantitative analysis of AS-YFP foci. The number of YFP foci in the head region (from the base of the second pharyngeal bulb to the nose) of C. elegans was plotted for a group of 15 animals each exposed to either curli-producing E. coli (Curli) or its corresponding mutant (Mutant). The horizontal lines represent the average number of AS-YFP foci. p<0.01, unpaired t test. (c) Congo red staining of C. elegans expressing AS-YFP exposed to curli-producing E. coli. Congo red stained deposits (arrowheads) in the head region of C. elegans expressing AS-YFP in the body wall muscles colocalized with AS-YFP aggregates. Scale bar=100 μm.

FIG. 4 shows plastic sections of retina from Group A (mutant E. coli lacking the curli operons); Group B (wild type E. coli producing curli); and Group C (control receiving only vehicle). Differences between the groups were not observed. The laminar organization of the retina appears similar in all groups. INL: inner nuclear layer; IPL: inner plexiform layer; ONL: outer nuclear layer; PRL: photoreceptor layer; RGCL: retinal ganglion cell layer.

FIG. 5 shows GFAP labeling of basal cells in rat cornea. Groups are as described for FIG. 4. Differences between the groups were not observed.

FIG. 6 shows Serum levels of IFN-γ and TLR-2. Sera were obtained from Control animals (cont, N=7), animals exposed to curli-producing wild type bacteria (w/curli, N=8), and animals exposed to bacteria unable to produce curli (w/o curli, N=9). Serum IFN-γ (A) and TLR-2 (B) were measured by ELISA assay. Data is expressed as O. D. value (mean±SE). All data are representative of two assays with similar results. There were no significant differences detected.

FIG. 7 shows a scatterplot matrix of microglial cell counts and synuclein optical densities for the two subgroups of unexposed (A—black and C—green) animals compared to exposed animals (B—red). Open and closed circles represent the first and second experimental replicates, respectively. For each measured variable, the exposed animals exhibited substantially greater evidence of inflammation than the two groups of unexposed animals. As for the scatterplot matrix, the labels down the diagonal indicate what the axes represent. Each column has the same variable on the x-axis and each row has the same variable on the y-axis. For example, the scatterplot in the third column of the second row has “u-glia frontal” on the y-axis and “u-glia hippo” on the x-axis. The different colors also represent the three groups, showing clear differences between group B (in red) and the others, while the filled vs unfilled circles distinguish subgroups 1 and 2 (which show no difference in any group). Original color figure in Chen et al. (2016), Supplementary Materials.

### DETAILED DESCRIPTION

All scientific and technical terms used herein, unless otherwise defined below, are intended to have the same meaning as commonly understood by one of ordinary skill in the art to which this invention belongs. In the case of any conflict, the present specification, including definitions, will control. References to techniques employed herein are intended to refer to the techniques as commonly understood in the art, including variations on those techniques or substitutions of equivalent or later-developed techniques which would be apparent to one of skill in the art. In order to more clearly and concisely describe the subject matter which is the invention, the following definitions are provided for certain terms which are used in the specification and appended claims.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the term “amyloid” means a form of a protein comprising aggregates of folded monomers which form polymeric fibrils. Typically, the folded forms include (3-sheet conformations of the protein.

As used herein, the term “amyloidogenic” means capable of forming an amyloid or causing formation of an amyloid.

As used herein, the term “proteinopathy” means a neurodegenerative disease characterized by the abnormal accumulation of amyloid protein aggregates in neurons, nerve fibers or glial cells.

As used herein, the term “alpha-synuclein” or “α-synuclein” means the human protein, also known as SNCA, NACP, PARK1, PARK4 and PD1, and typified by the amino acid sequences disclosed in protein databases as UniProt P37840, NP_000336, NP_001139526, NP_001139527 and NP_009292, as well as mammalian homologues and allelic variants thereof (e.g., A30P and A53T). In the context of the present invention, mutant α-Syn allelic variants associated with the development of a-synucleinopathies are of particular interest, and artificial α-Syn variants that substantially retain α-Syn biological activities can be employed in the assays of the invention. The term “α-Syn” specifically includes variants which are known to increase oligomerization, including the following: A30P, E35K, E46K, H50Q, A53T and E57K. See, e.g., Tsigelny et al. (2015), ACS Chem Neurosci. 6(3): 403-416.

As used herein, the term “alpha-synucleinopathy” or “α-synucleinopathy” means a neurodegenerative disease characterized by the abnormal accumulation of the aggregated or amyloid form of alpha-synuclein in neurons, nerve fibers or glial cells. There are over 50 alpha-synucleinopathies (see, e.g., McCann et al. (2014), “α-Synucleinopathy phenotypes,” Parkinsonism Rel. Disord. 20:S62-7), of which PD is the most common and most widely studied.

As used herein, the term “curli” means the major proteinaceous component of the extra-cellular matrix produced by many Enterobacteriaceae, including E. coli and Salmonella spp., and is involved in adhesion to surfaces, cell aggregation, and biofilm formation. CsgA is the major curli protein subunit, and curli fibers consist primarily of long polymers of CsgA which are nucleated by CsgB. Curli fibers belong to the class of bacterial amyloids. See, e.g., Barnhart & Chapman (2006), Annu. Rev. Microbiol. 60:131-147. Nucleic acid and amino acid sequences for the E. coli CsgA gene and protein can be found at, for example, GenBank Accession OYK48210.1.

As used herein, the term “gastrointestinal tract” means the interior or luminal portion of the organ system which extends from the mouth to the anus. As used herein, the gastrointestinal tract includes the oral cavity, nasopharyngeal cavity, esophagus, stomach, small intestine (duodenum, jejunum and ileum) large intestine (cecum, colon, rectum, and anal canal).

As used herein, the term “prebiotic” means a consumable composition (e.g., food, food supplement, food component) that induces the growth or activity of beneficial or commensal microorganisms in the human gastrointestinal microbiome.

As used herein, the term “probiotic” means a composition of live commensal microorganisms (e.g., bacteria) that induces a healthful or beneficial effect in the human gastrointestinal tract. A probiotic may contain, for example, a sample of wild-type strains of one or more species of commensal bacteria that inhabit the normal, healthy human gut, such as Bacteroides, Porphyromonas, Bifidobacterium, Lactobacillus, and Clostridium species.

As used herein, the term “exposure” means presence in the same medium such that direct physical contact may occur by diffusion. For example, adding a soluble compound to a fluid medium including a cell entails exposure of the cell to that compound. Similarly, administering a compound to a body such that, by some combination of active transport and diffusion, the compound can make direct physical contact with a cell entails exposure of the cell to that compound.

The term “antibody” or abbreviation “Ab,” as used herein, includes whole antibodies and any antigen binding fragment or derivative thereof (i.e., “antigen-binding domain”), with or without native glycosylation. The term “antigen-binding domain” of an antibody, as used herein, refers to one or more domains of an antibody that retain the ability to specifically bind to an antigen. Examples of antigen binding domains encompassed within the term “antigen-binding domain” of an antibody include a Fab fragment, F(ab′)₂ fragment, Fab′ fragment, Fd fragment, Fv fragment, scFv fragment, dAb fragment, heavy-chain only antibodies (HCAbs), diabodies and nanobodies, as well as multivalent and multispecific versions thereof

As used herein, the term “reduce” means to cause any statistically significant reduction in level or activity when compared to a control, base-line or reference state . Such a reduction may be up to 5%, 10%, 20%, 30%, 40%, 50%, 60%, 70%, 80%, 90%, 95%, or up to 100%. Accordingly, the term “reduced” encompasses both a partial reduction and a complete elimination of the level or activity.

As used herein, the term “wild-type” means the form of a microbe or protein that is most prevalent in a natural population, as well as genetic variants that do not substantially alter the phenotype with respect to the relevant biological characteristics of the wild-type. Thus, a population may have one or more wild-type genotypes which have insubstantial differences with respect to particular functions. In contrast, a mutant “form” of a microbe or protein has substantially different phenotype or activity than a wild-type form, including substantial differences in pathogenicity or other relevant characteristics.

### Incorporation by Reference

The patent, scientific and technical literature referred to herein establish knowledge that was available to those skilled in the art at the time of filing. The entire disclosures of the issued U.S. patents, allowed applications, published and pending patent applications, and other references, including database citations for nucleic acid and protein sequences, that are cited herein are hereby incorporated by reference to the same extent as if each was specifically and individually indicated to be incorporated by reference.

### Principles of the Invention

The present invention depends, in part, upon the demonstration that bacterial amyloid proteins present in the gastrointestinal tract are capable of inducing the first steps in the development of amyloid protein disease in the central nervous system. This has been demonstrated by experiments in an animal model in which amyloid deposits in the gut and neurons in the brain were measured and compared to controls. These experiments, described in detail below, demonstrated that animals receiving oral administration of bacteria producing a bacterial amyloid protein exhibited greater human amyloid deposits in the gut and brain neurons than animals receiving oral administration of control bacteria incapable of producing the bacterial amyloid. Additional experiments in the roundworm Caenorhabditis elegans support these findings. This is believed to be the first demonstration of causation of amyloidogenic neurodegenerative disease symptoms in an animal by amyloid proteins produced by bacteria in the gastrointestinal microbiome.

Thus, to evaluate the role of amyloid proteins made by the microbiota, aged rats and transgenic C. elegans were exposed to E. coli producing the extracellular bacterial amyloid protein curli. Rats exposed to curli-producing bacteria displayed increased neuronal alpha-synuclein (AS) deposition in both gut and brain and enhanced microgliosis and astrogliosis compared to rats exposed either to mutant bacteria unable to synthesize curli, or to vehicle alone. There were no differences among the rat groups in survival, body weight, inflammation in the mouth, retina, kidneys or gut epithelia, and circulating cytokine levels. AS-expressing C. elegans fed on curli-producing bacteria also had enhanced AS aggregation. These results suggest that bacterial amyloid functions as a trigger to initiate AS aggregation through cross-seeding and also primes responses of the innate immune system. Details of these experiments are described in the examples below, as well as Chen et al. (2016), Sci. Rep. 6:34477, published after the priority date of the present application.

The experiments showed that exposure to bacteria producing a functional extracellular amyloid protein enhances aggregation of AS in brain neurons in aged rats and in muscle cells in nematodes. In gut neurons, the exposure increased levels of AS in the submucosa. The mechanism of these effects may involve upregulation of protein expression, which is well known to enhance aggregation. These effects can also have resulted from cross-seeding, which has been reported between bacterial amyloids, including curli, and mammalian proteins (24). Lee and coworkers have demonstrated that AS aggregates seed aggregation of tau. PrPsc has been reported to seed AB aggregation and curli seeds aggregation of serum amyloid A (25). However, there have been no studies on how cross-seeding by exogenous bacterial amyloid affect the propagation and aggregation of endogenous amyloid proteins in living animals.

The finding of enhanced AS deposition without aggregation in the gut but with aggregation in the brain is of interest, and may indicate that the period of exposure was too short to allow for polymerization of AS in gut neurons. It is also possible that the biophysical factors involved in induction of AS aggregation and the relationship between AS expression and aggregation are different in the gut and brain (due to pH and other factors). Because the bacteria were delivered orally, the pathway for induction of changes in the brain can involve the autonomic nervous system, particularly the vagus and other nerves. This is the route which is believed to be responsible, at least in part, for transmission of bovine spongiform encephalopathy and related prionoses (7). Recent studies have also suggested the involvement of the vagus nerve in PD (26). Brain alterations can also have been induced through oral tissues and their rich innervation as well as via a hematogenous route.

C. elegans is a genetically tractable organism for elucidating the critical events mediating interactions between bacterial amyloid proteins and AS in intestinal tissues and potential propagation of AS aggregates to neurons (27). The finding that AS aggregation in worms is enhanced by exposure to curli expressing bacteria can also involve a cross-seeding mechanism. Since nematodes naturally take up microorganisms as food, they allow for mechanistic studies of cross-seeding by different microbial amyloid proteins expressed by distinct strains during feeding and throughout their lifespan.

The development of microgliosis, astrogliosis and enhanced expression of IL-6, TLR2 and TNF in the brain following curli exposure suggests the occurrence of an enhanced local sterile inflammatory response to AS in the brain. These findings do not appear to be caused by T cell activation by bacterial amyloid, as cellular infiltrates were not found in the brain or other tissues. Activation of the immune system in both AD and PD have now been extensively established (28). The importance of the immune system in AD was first proposed by Braak and colleagues (6), and has been recently supported by the association of immune system genes involved in neurodegeneration (for recent review see Colonna (14)). It has been shown that impaired microglial proliferation slows AD in Tg mice (29). Bacterial amyloid is recognized as a pathogen associated molecular pattern (PAMP) with messengers including TLR2/1, CD14, NFkB and iNOS. These messengers of the innate immune system are involved in its recognition of AS, AB and also curli. Recently it has been shown that TLR2 inhibition prevents AS aggregation through activation of autophagy (20), suggesting that TLR2 activation through exposure to bacterial amyloid is pathogenic. Microglial TLR2 activation has also been reported to increase cellular uptake of AB and is involved in AB stimulated microglial activation (30). The co-receptor molecule CD14 has also been linked to oxidative damage and dendritic degeneration following innate immune system activation (31).

Priming of immune cells to respond to these bacterial or other amyloids in the gut may cause enhanced responses to neuronal amyloids in the brain. Gallo et al. (32) have recently observed that bacterial amyloid binds to bacterial and eukaryotic DNA and causes amyloid polymerization, as well as enhancement of autoimmunity. The intensity of the immune system's response to cerebral amyloid deposition is important in determining the development of dementia in AD and PD, as many older persons have been shown to have significant AB pathology without dementia and without microglial activation (17).

The precise roles of protein aggregation and inflammation in neurodegeneration remain unclear (33). Aggregation of AS and production of toxic oligomers are pathogenic, but intracellular AS aggregation into fibrils can be protective (34). Furthermore, it has been shown in studies of the molecular machinery for the production of curli, that the same protein accelerated the aggregation of one protein and inhibited the amyloid formation of another (35). There is also evidence that an active immune response is neuroprotective, as suggested by the work on immunotherapy for AD. Immune responses participate in clearance of AB plaques and improved cognition in animal studies (36) and microglia produce a barrier reducing the toxic effects of AB on neurotic dystrophy (37). It has been proposed that the association of AD and PD with age is related to senescence of the immune system and lowered immune responses (38).

There is now a vast literature documenting the influence of the microbiota on metabolism, immunity, cancer, diabetes, obesity, intestinal diseases, heart disease and other conditions. Bacteria, fungi and other organisms comprising our microbiota make functional amyloid proteins. Biofilms are found in the body and up to 40% of bacterial biofilms have amyloids (9). Evidence for a role for fungi in AD has recently been proposed, but the possible role for fungal amyloids has not yet been considered (41). Although it is now documented that many bacteria that make amyloid proteins are components of human microbiota, the presence of these proteins in the body has not been comprehensively evaluated (32). Although E. coli is not a major component of human microbiota, there are several others that are important commensal partners, including Streptococcus mutans, Staphylococcus aureus, Salmonella enterica, Mycobacterium tuberculosis and others (8, 42). Gene homologs encoding curli were recently determined also in four phyla: Bacteroidetes, Proteobacteria, Firmicutes, and Thermodesulfobacteria (8). It is possible that the various amyloids to which humans are exposed influence misfolding of endogenous proteins with strain specificity, as has been reported for AS, AB and related proteins. That is, certain bacterial amyloids are particularly important for inducing aggregation of certain strains of a neurodegenerative disease protein, and this is reflected in distinct disease phenotypes (43, 44).

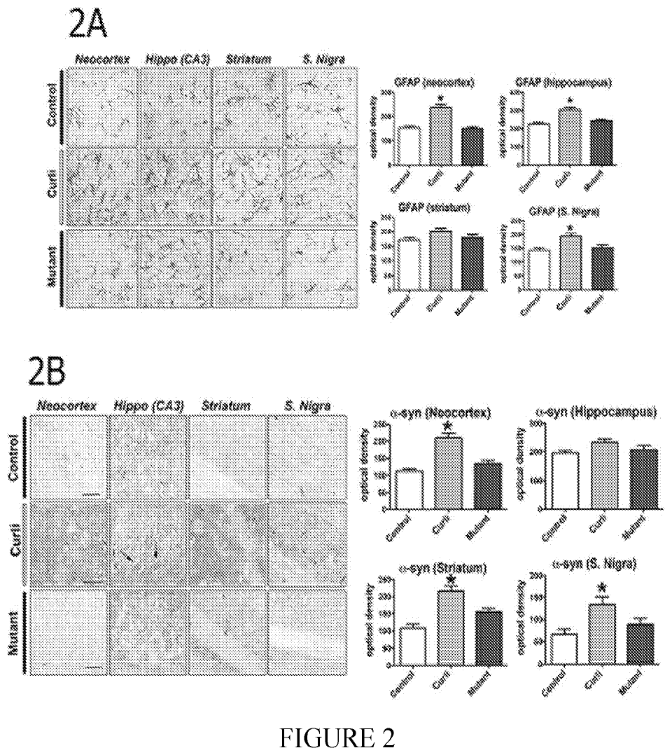

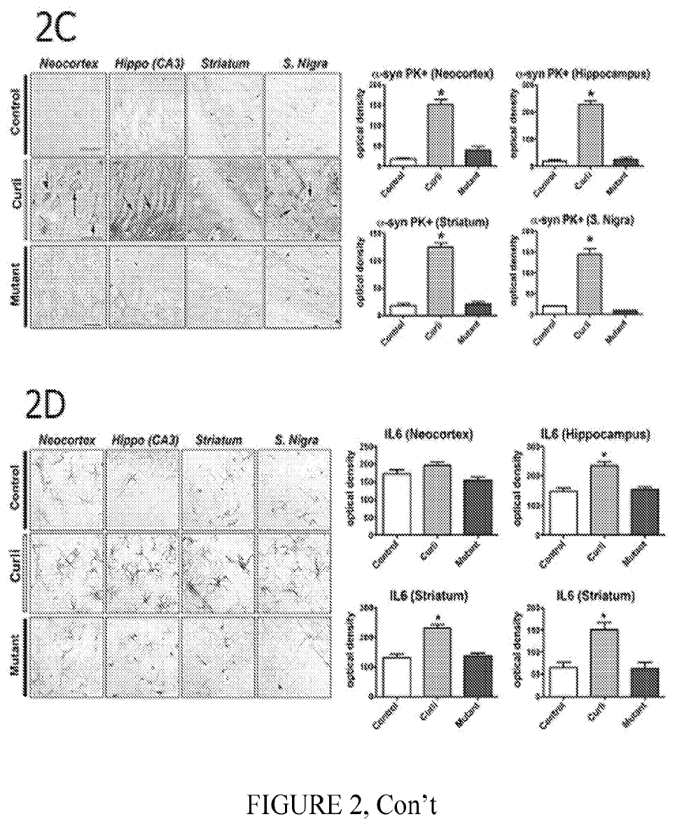

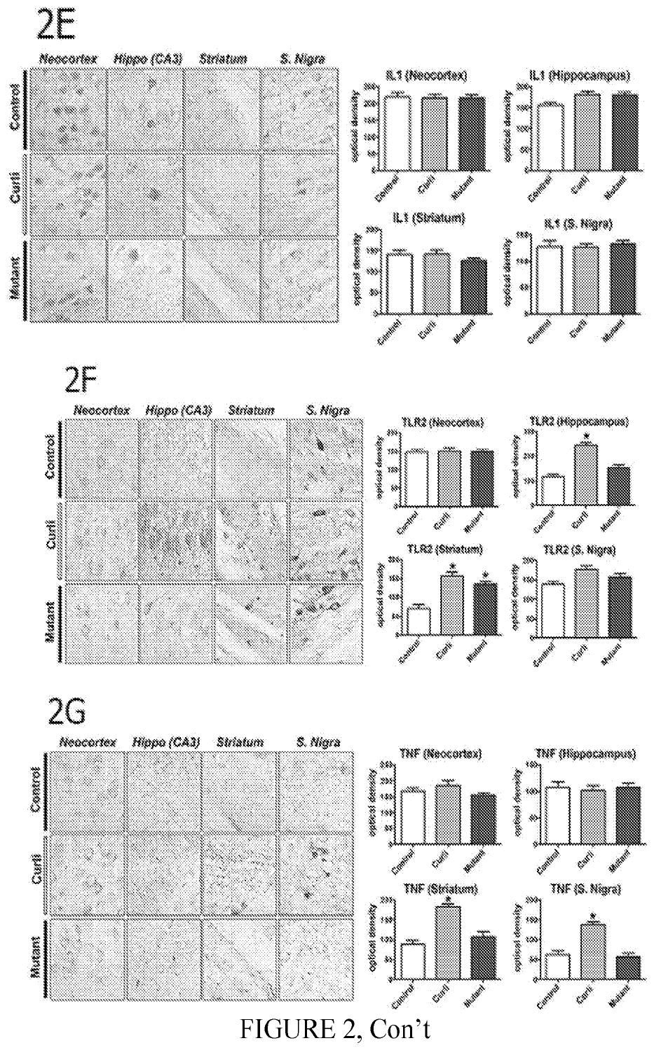

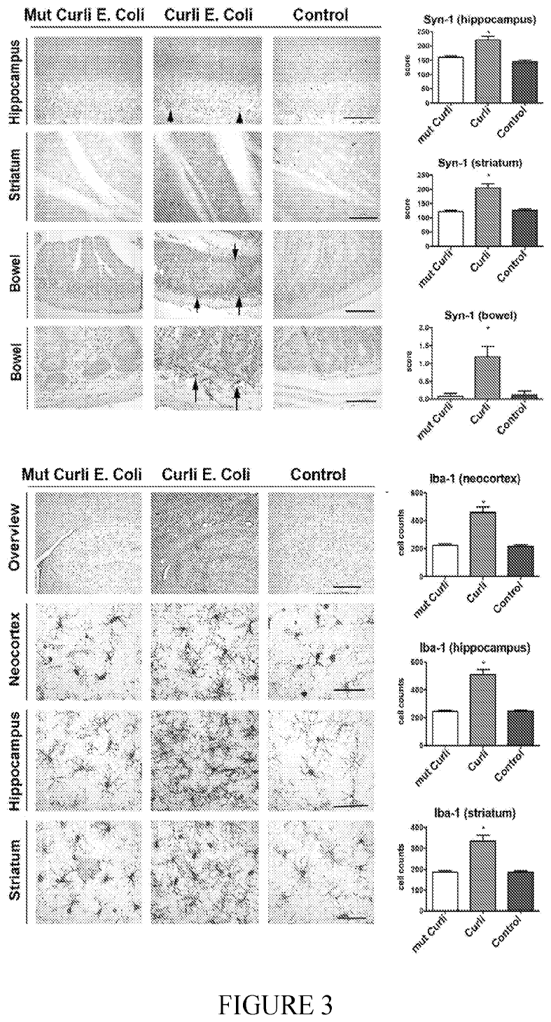

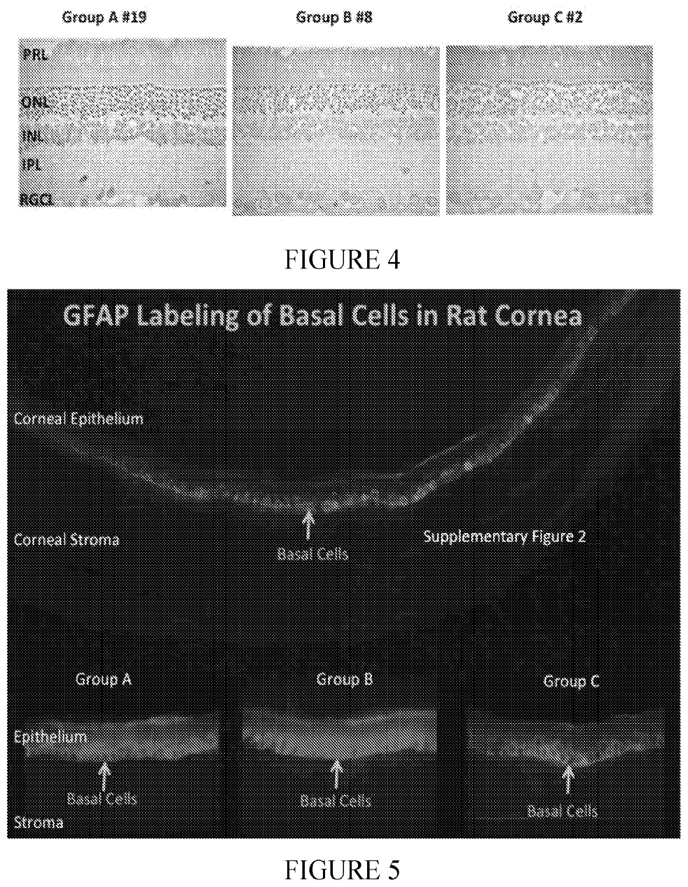

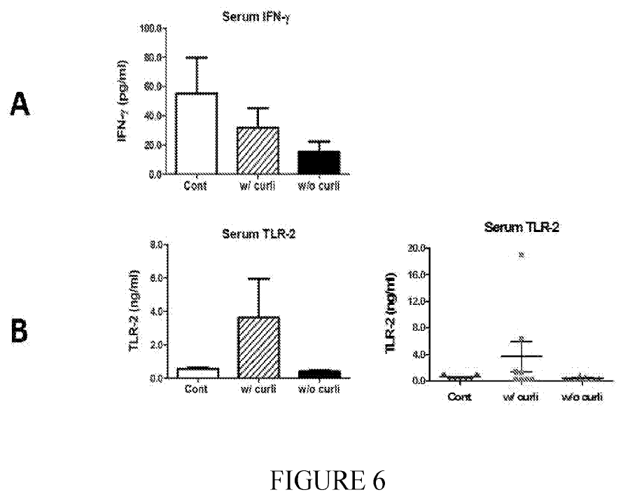

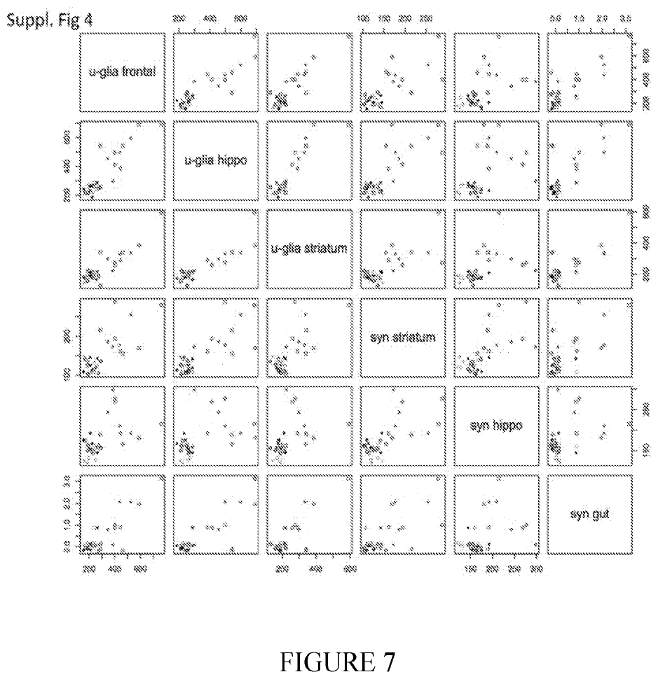

The experiments suggest that protein misfolding and immune activation in neurodegenerative disorders are triggered through cross-seeding by exposure to exogenous microbial amyloids in the nose, mouth and gut. Cross-seeding of amyloidogenic proteins by bacterial amyloids has been documented in both in vivo (12) and in vitro (24) (e.g., curli can cause cross-seeding of serum amyloid A (12)). We provide evidence for our proposed mechanism for the induction of neuroinflammation in the brain: the innate immune system utilizes TLR2 to recognize bacterial amyloir, and we demonstrate upregulation of TLR2 in the striatum and hippocampus of animals exposed to bacteria producing curli (FIG. 2F).

The experimental results disclosed herein (and also published after the priority date hereof as Chen et al. (2016), Sci. Rep. 6:34477) are amongst the first studies that evaluate the influence of bacterial amyloid on disease processes in living animals (40). The data indicate that amyloid proteins in the microbiota are involved in the origination and maintenance of neurodegenerative disease.

### EMBODIMENTS OF THE INVENTION

In one aspect, the invention provides methods of preventing, inhibiting and/or decreasing protein deposits of a human amyloid protein and/or neuroinflammation in the central nervous system by reducing an amount of exposure to a pathogenic bacterial amyloid in the gastrointestinal tract.

In another aspect, the invention provides methods of preventing, inhibiting and/or treating a proteinopathy by reducing an amount of exposure to a pathogenic bacterial amyloid in the gastrointestinal tract.

In some embodiments, the methods are limited to use in human subjects or patients at risk of (e.g., diagnosed with a condition associated with a neurodegenerative proteinopathy, such as IBD; or testing positive for a biochemical marker of a neurodegenerative proteinopathy), or diagnosed with a neurodegenerative proteinopathy, or showing symptoms of a neurodegenerative proteinopathy (e.g., motor or cognitive deficits).

In general, the methods are considered to prevent amyloid protein deposits or a proteinopathy if, in a population of subjects, the development of amyloid deposits and/or proteinopathy is expected in a certain percentage of the subjects, and the method prevents the development of the amyloid deposits or proteinopathy in some of those subjects, reducing the percentage of affected individuals below the expectation. For example, there is a well-known statistical correlation between inflammatory bowel disease and the development of PD. The methods of the invention can be used to treat the population of IBD patients, and will prevet PD in some of them, reducing the percentage that become affected, and the correlation. Similarly, the methods of the invention can be used prophylactically to prevent the development of amyloid deposits and proteinopathies in patient populations with a variety of risk factors or early signs of these conditions.

Similarly, the methods of the invention are considered to inhibit amyloid protein deposits or proteinopathy if, in a population of subjects, the progression of the proteinopathy (e.g., increased or more extensive amyloid deposits, progression of deposits from the vagus nerve to the CNS, development of neurological deficits) is expected at a certain rate, to a certain degree, or in a certain percentage of the subjects, and the method reduces the rate of progression, the degree of progression, or the percentage of affected individuals below the expectation.

Finally, the methods of the invention are considered to treat amyloid protein deposits or proteinopathy if, in a population of subjects, the development of amyloid deposits and/or symptoms of proteinopathy is reversed in at least some subjects such that the percentage of affected individuals is reduced below expectations.

In some embodiments, the methods of the invention are limited to use in human subjects or patients at risk of (e.g., diagnosed with a condition associated with a neurodegenerative proteinopathy, such as IBD; or testing positive for a biochemical marker of a neurodegenerative proteinopathy), or diagnosed with a neurodegenerative proteinopathy, or showing symptoms of a neurodegenerative proteinopathy (e.g., motor or cognitive deficits).

In some embodiments, the human amyloid protein is alpha-synuclein (AS), amyloid beta (AB), Tau, FUS or TDP43. In other embodiments, other amyloid proteins associated with proteinopathies arising in the gut are included. The human amyloid proteins in each embodiment can include both wild-type proteins and mutants to confer increased likelihood of pathogenesis (e.g., alpha-synuclein mutants such as A30P, E35K, E46K, H50Q, A53T and E57K).

In some embodiments, the pathogenic bacterial amyloid is an amyloid produced by a bacterium of a phylum selected from the group consisting of Bacteroidetes, Proteobacteria, Firmicutes and Thermodesulfobacteria. In some of these embodiments, the pathogenic bacterial amyloid is the E. coli curli protein (or the E. coli A protein subunit) or a Bacteroidetes, Proteobacteria, Firmicutes or Thermodesulfobacteria homologue of the E. coli curli protein or the E. coli A protein subunit.

In some embodiments, the reduction in exposure is accomplished by administering at least one antibiotic to reduce the relative abundance of the bacterial strain producing the pathogenic bacterial amyloid. In such embodiments, the antibiotic can be specific for the pathogenic bacterial strain in question. Unfortunately, there are few if any antibiotics with such specificity and, therefore, antibiotic therapies are likely to kill many beneficial bacteria along with the targeted pathogens. Nonetheless, such a therapy can be medically justified, and adjunct therapy (e.g., probiotic therapy) can be in combination to help reconstitute a healthy gastrointestinal flora after the antibiotic therapy. In some instances, it may be possible to use antibody-based or nucleic acid-based therapeutics (e.g., nanobodies or siRNA technology) to target certain populations of microbes.

In some embodiments, the reduction in exposure is accomplished by administering at least one prebiotic to increase the relative abundance of non-pathogenic bacterial strains which will at least partially displace the bacterial strain producing the pathogenic bacterial amyloid. In these embodiments, prebiotics that enhance the growth, survival or adhesion of non-pathogenic (or even beneficial) bacterial strains can alter the composition of the gastrointestinal microbiome to reduce the relative abundance of the pathogenic strains and thereby ameliorate the effects of the pathogenic bacterial amyloid. For example, there is evidence that high fiber diets may increase the relative abundance of some Bacteroidetes spp. while decreasing some Firmicutes spp.

In some embodiments, the reduction in exposure is accomplished by administering at least one prebiotic to reduce the relative abundance of the bacterial strain producing the pathogenic bacterial amyloid relative to a non-pathogenic bacterial strain which will at least partially displace the pathogenic strain. In these embodiments, prebiotics that inhibit the growth, survival or adhesion of pathogenic bacterial strains can alter the composition of the gastrointestinal microbiome to reduce the relative abundance of the pathogenic strains and thereby ameliorate the effects of the pathogenic bacterial amyloid. For example, there is evidence that certain galactooligosaccharides may reduce adherence and decrease the relative abundance of some pathogenic Escherichia coli strains.

In some embodiments, the reduction in exposure is accomplished by administering probiotics to provide non-pathogenic bacterial strains which will at least partially displace the bacterial strain producing the pathogenic bacterial amyloid. Such probiotics may, for example, include wild-type, non-pathogenic strains of bacteria that are commonly found in the human gastrointestinal microbiome, such as Faecalibacterium prausnitzii, Peptostreptococcus spp., Peptococcus spp., Bacteroides fragilis, Bacteroides melaninogenicus, Bacteroides oralis, Enterococcus faecalis, Escherichia coli, Enterobacter spp., Klebsiella spp., Bifidobacterium bifidum, Staphylococcus aureus, Lactobacillus spp., Clostridium perfringens, Proteus mirabilis, Clostridium tetani, Clostridium septicum, Pseudomonas aeruginosa, Salmonella enterica, and others known in the art.

In some embodiments, the reduction in exposure is accomplished by administering a pharmaceutical preparation with an active agent that alters the pathogenic bacterial amyloid to a non-pathogenic form (e.g., a disaggregated or other form incapable of cross-seeding a human amyloidogenic protein). For example, an active agent that disrupts curli protein polymers on E coli can disrupt the initiator of alpha-synuclein aggregation and/or negatively affect E. coli biofilms in the gut such that the bacteria are cleared more easily and are reduced in relative abundance.

In some embodiments, the reduction in exposure is accomplished by administering a pharmaceutical preparation with an active agent that alters the interaction between the pathogenic bacterial amyloid and a human amyloidogenic protein such that nucleation of the aggregated form is inhibited, prevented and/or reversed. For example, an active agent that blocks the interaction (e.g., by competitive binding or allosteric inhibition) between an E. curli protein (either as a monomer or amyloid aggregate) and an alpha-synuclein monomer can prevent initiation of alpha-synuclein aggregates by the curli protein. Antibodies, including nanobodies, against the curli amyloid or alpha-synuclein monomer could block such interactions.

In some embodiments, the reduction in exposure is accomplished by administering a pharmaceutical preparation with an active agent that alters the interaction between the pathogenic bacterial amyloid and cells of the human gastrointestinal tract such that nucleation of the aggregated form of a human amyloidogenic protein is inhibited, prevented and/or reversed. In some specific embodiments, the interaction is uptake of the pathogenic bacterial amyloid into cells of the human gastrointestinal tract. For example, if the curli protein (either as a monomer or amyloid aggregate) is actively transported across the cell membranes of the gastrointestinal tract, an inhibitor of that transport can prevent curli from reaching alpha-synuclein monomers in the gastrointestinal tract or enteric nervous system, and prevent or inhibit the initiation of the amyloid protein pathogenesis.

In some embodiments, the reduction in exposure is accomplished by administering a pharmaceutical preparation with an active agent which immunizes a subject against the pathogenic bacterial amyloid or pathogenic bacteria such that the subject's immune system reduces the relative abundance of the pathogenic bacteria. For example, immunization with bacterial amyloid monomers or whole bacterial cells fibrils can lead to development of a humoral immune response. Antibodies against the bacteria or their amyloid proteins could reduce the amount of amyloid available for interaction with the cells of the gastrointestinal tract, and prevent, inhibit or treat the corresponding proteinopathy. In some embodiments, the reduction in exposure is accomplished by orally or otherwise administering a pharmaceutical preparation with an active agent which decreases production of bacterial amyloid. In some embodiments, such administration of an active agent that decreases production of bacterial amyloid is therapeutic or preventative against a neurodegenerative disease without entry into the blood or brain.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

### EXAMPLES

This disclosure is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Exposure to Curli-Producing Bacteria Enhances AS Aggregation in Rat Brain and AS Deposition in Gut

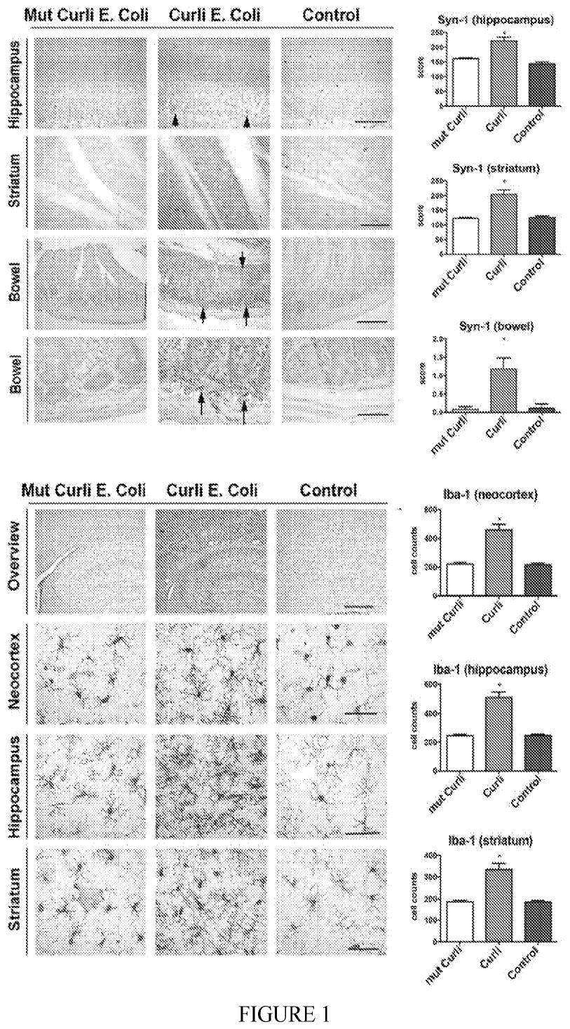

Aged Fischer 344 rats have been described to have aggregated AS in the intestinal submucosal plexus (21). These animals were used to evaluate the influence of exposure to bacteria producing amyloid proteins on AS deposition and aggregation in the gut as well as in the brain. Animals were exposed weekly via the oral route using an established method for bacterial exposure (22). Rats exposed to wild-type bacteria producing the amyloid protein curli had enhanced AS deposition in gut ganglion cells (myenteric plexus and submucosa) (FIG. 1a, top panel). Additionally, rats exposed to wild-type bacteria producing curli had enhanced AS deposition in neurons in hippocampus (especially CA3) and striatum, as compared to rats exposed to mutant bacteria lacking the capacity to produce curli or to rats exposed only to vehicle (FIG. 1a). Deposits in neurons in the brain were proteinase K resistant, indicating the presence of aggregated AS (FIGS. 2b and c). Quantitation showed that rats exposed to curli-producing bacteria had more AS deposition in the gut than the other 2 groups with an odds ratio of 2.5 (95% CI 4.4-239) demonstrating that exposed animals had a higher probability of having higher gut scores than unexposed animals (lower panel, FIG. 1a and FIG. 7). Alpha-synuclein aggregation was not related to length of exposure (2 or 3 months). AS deposits in the gut were proteinase K sensitive, suggesting the absence of AS aggregation in the gut.

### Exposure to Curli-Producing Bacteria Enhances Immune Responses in Rat Brain

AS as well as the bacterial amyloid protein curli are recognized by the innate immune system with involvement of similar pathways (TLR2, CD14, NFkB, iNOS). Therefore markers of neuroinflammation were assessed, including microgliosis, astrogliosis and the pro-inflammatory cytokines interleukin-6 (IL-6), interleukin-1 (IL-1) tissue necrosis factor (TNF) and TLR2, to determine the immune responses in the brain of exposed animals. Microgliosis (Iba-1, allograft inflammatory factor, FIG. 1b), as well as astrogliosis (GFAP, glial fibrillary acidic protein, FIG. 2a) in the striatum, hippocampus and neocortex was significantly higher in animals exposed to curli-producing bacteria as compared to the two control groups. Interleukin-6 (IL-6) expression was also higher in the hippocampus, stratum and substantia nigra of the animals exposed to curli than in the control groups (FIG. 2d). Toll like receptor 2 (TLR2) expression was also higher in the hippocampus and striatum in animals exposed to curli-producing bacteria as compared to the other 2 groups (FIG. 20 and TNF expression was higher in striatum and substantia nigra of animals exposed to curli-producing bacteria than that in the other 2 groups (FIG. 2g). There were no significant differences in interleukin 1 expression (FIG. 2e). The results were not related to length of exposure.

### Observed Phenotypes Were Not Related to Illness, Aging or Cellular Immune Responses.

Body weight, evidence of inflammatory infiltrates, and survival were assessed to determine the possibility that the effects of exposure noted above were caused by illness induced by the bacteria. There were no significant differences among the 3 groups in terms of survival, body weight or cellular inflammation in oral tissues, kidneys, eyes, brain or gut. Examination of the retina in 12 eyes (4 eyes from each group) was performed using standard histology and immunohistochemistry, as the eyes are located in proximity to oral tissues. The stratification of the retinal layers appeared similar across all eyes and groups. Frozen retinal sections (20 μm thickness) immunolabeled with GFAP (labeling astrocytes and activated Muller cells) and vimentin (labeling Muller cells) were similar across all eyes and groups (see below).

### Serum Cytokine and TLR-2.

Serum levels of several cytokines and related molecules were evaluated to gain an insight into the systemic immune responses. Serum levels of IL-1β, IL-6, IL-10, and TNF-α were below detection levels. Serum INF-y was detectable but there was no difference among three groups. Serum TLR2 levels were trending higher in the rats exposed to curli than the other two groups but the differences did not reach statistical significance (FIG. 6). Therefore, no conclusions concerning the presence of systemic immune responses to the exposures was made.

AS-Expressing C. elegans With Curli Bacteria Displayed Increased AS Aggregates.

AS expressing C. elegans were exposed to wild-type E coli expressing curli and mutant E. coli lacking the ability to synthesize curli, to evaluate the influence of bacterial amyloid on AS aggregation in another organism. The nematode C. elegans naturally feeds on E. coli, and protein aggregation can be studied in vivo thus representing an attractive model organism to study how bacterial exposures influences AS aggregation. A transgenic (Tg) C. elegans line expressing human AS fused with YFP (AS-YFP) in body wall muscle was used, allowing for visualization of aggregated AS-YFP in live animals using fluorescence microscopy (23). As shown in FIG. 3a, AS-expressing C. elegans fed with curli-producing E. coli displayed increased AS deposits compared to those fed with the mutant E. coli lacking curli production. The Congo red stained deposits coincide with those of AS-YFP, confirming that AS-YFP forms amyloidogenic aggregates recognized by Congo red (FIG. 3b). AS-Aggregates accumulated in the head first and moved to the tail during adulthood. Live worms were stained with the cytosolic oxidative stress marker CellRox Red, but no difference was seen between worms exposed or not exposed to curli-producing bacteria. Suggesting that enhanced AS aggregation was not due to increased oxidative stress in the cytosol. A mitochondrial oxidative stress marker MitoSox Red was also tested, but only weak staining was detected in both groups. Swimming tests were also performed to evaluate locomotive activity and showed a trend toward decreased thrashing rate (˜15-20%) in worms exposed to wild-type curli-producing E. coli vs. mutant E. coli lacking curli, which did not reach statistical difference.

### Antibodies and Chemicals

The protease and phosphatase inhibitor cocktails and Congo red were purchased from Sigma-Aldrich (St Louis, Mo.). Hypoestoxide was obtained from Immune Modulation, Inc. (Bloomington, Calif.). The following antibodies were used: α-synuclein (Syn-1; BD Bioscience, San Diego, Calif.); TNFα, glial fibrillary acidic protein (GFAP) (GA5), TH, and NeuN (Millipore, County Cork, Ireland); β-actin (Sigma-Aldrich, St Louis, Mo.); NF-κB p65 and phospho-NF-κB p65 (Cell signaling, Beverly, Mass.); IL-1β and IL6 (Abcam, Cambridge, Mass.); α-synuclein (CT, Syn105) [18]; α-synuclein (syn211) (Life Technologies, Grand Island, N.Y.); and Iba-1 (Wako, Richmond, Va.).

### Animals

Aged male Fischer 344 rats were obtained from the National Institute of Aging, NIH (M. Murthy). These rats were chosen because it has been described that their neurons in the gut develop deposits of AS with age, thus mimicking PD (46). At the time of euthanasia via CO2 asphyxiation the rats were 22.5-25 months of age. All animal procedures were approved by the Institutional Animal Care and Use Committee of the University of Louisville (Protocol 12022) and were also in compliance with United States Public Health Service standards and National Institutes of Health guidelines. The University of Louisville Animal Care Facility is accredited by AAALAC International.

### Bacteria Preparation and Exposure

A wild-type E. coli strain LSR12 and the isogenic mutant lacking both curli operons (strain C600 with deletion of the curli operons) were obtained through the generosity of M. Chapman, University of Michigan. The identity of strains has been verified using PCR with oligonucleotides priming at individual curli genes and spanning the entire curli region (oligonucleotide sequences are available upon request). Congo Red (CR) staining was used to confirm that wild-type strain is CR-positive while curli mutant is CR-negative (data not shown). The bacteria were otherwise identical. E. coli was grown in TSBYE which consists of 30 grams per liter trypticase soy broth (Difco) supplemented with 2% (w/v) yeast extract, 1 mg/ml hemin (final concentration), and 5 mg/ml menadione (final concentration) (10% CO₂, 10% H₂, and 80% N₂) at 37° C. for 48 hours.

Oral exposure of rats was performed essentially as previously described by Baker et al (47). A total of 11-13 rats per group were used per experiment. Animals were initially treated with sulfamethoxazole (MP Biomedical, Solon, Ohio) at a final concentration of 800 mg/ml and trimethoprim (Sigma, St. Louis, Mo.) at a final concentration of 400 mg/ml ad libitum for 10 days at 2 day intervals. Four days after the last antibiotic treatment, the rats began oral exposure suspended in 1 ml 2% carboxymethylcellulose (CMC; MP Biomedical, Solon, Ohio) in sterile PBS using a 2.25 mm feeding needle (Popper and Sons, Inc., New Hyde Park, N.Y.). Animals were inoculated orally every week for 2 months (N=19) or 3 months (N=14). Animals were studied in 3 groups: Vehicle only (N=13); exposure to wild type bacteria (curli-producing) (N=11); and exposure to mutant bacteria (lacking curli operon) (N=9). Initially the rats were subjected to antibiotic treatment in water for 10 days. This was followed by no antibiotic treatment for seven days. The bacteria were propagated and bacterial slurry was prepared by 1:1 dilution of the working bacterial stock solution with 4% carboxy methyl cellulose (CMC) such that the working bacterial stock was 1×10¹⁰ cfu/ml. The vehicle control group was treated with 1:1 dilution of CMC in equal volume of Krebs+ buffer. The appropriate bacteria were administered in the mouth of the rats in order to expose the gingiva as well as to colonize the gut. Rats were weighed several times each month. Bacteria were administered orally every week for 2 or 3 months. At the end of 2-3 months rats were sacrificed and various organs and blood were collected and banked.

### Serum Cytokine Analyses

Blood was collected from the hearts of the aged rats treated with or without curli-producing E. coli, and control rats without any treatments (N=7-8 per group). The blood samples were centrifuged at 4° C. (1,000×g) for 30 min to extract the serum. The sera were stored in aliquots at −80° C. until use. ELISAs were performed according to the manufacturer's protocol. Serums IL-1β, IL-6, IL-10, INF-γ, and TNF-α were measured by R&D Systems Quantikine ELISA kits and serum TLR-2 was determined by an ELISA kit from Antibodies-Online (Atlanta, Ga.). Data were expressed as optical density (O.D.) value at 450 nm with double wells per sample and the wavelength correction was set at 570 nm. A standard curve was created for each tested cytokine at each time to determine the concentration of the target cytokine concentration in each sample. The concentration of the positive control sample was within the linear range of the standard curve. Results presented as means ±SE, and statistical analysis was done using GraphPad Prism software. One-way analysis of variance (ANOVA) with Turkey post-hoc test was used for multiple comparisons among groups. p<0.05 was considered statistically significant. Positive controls were tested to confirm the function of the assays.

### Immunocytochemistry

The neuropathological studies were carried out on blinded specimens. The procedures for immunohistochemical, immunofluorescence, and neuropathological analysis have been described elsewhere (55). Briefly, the right hemibrains were post-fixed in phosphate-buffered 4% paraformaldehyde at 4° C. for neuropathological analysis, blind-coded sagittal brain sections were incubated with primary antibodies at 4° C. for overnight following serial sectioning in the sagittal plane at 40 μm with a Vibratome 2000 (Leica, Deerfield, Ill.) for neuropathological and immunocytochemical analysis. The next day, sections were incubated with either biotinylated- or FITC-conjugated secondary antibodies and detected with avidin D-HRP HRP (ABC elite, Vector Laboratories, Burlingame, Calif.) and with Tyramide Signal Amplification Direct system (PerkinElmer, Waltham, Mass.), respectively. Analysis of AS accumulation was performed using free-floating, blind-coded sections (48). Brain sections were stained with Iba-1, GFAP, TNFα, IL-1β, IL6, human α-synuclein, NF-κB, and phosphorylated NF-κB antibodies, respectively to determine the neuroinflammation, neurodegeneration, accumulation of α-synuclein, and NF-κB activation. Sections were imaged by Olympus BX41 microscope. All immunoreactivity levels were determined by optical density analysis using Image Quant 1.43 program (NIH) except the immunoreactivity of Iba-1. The cell numbers of Iba-l-positive cells were determined per field (230 μm, Å˜184 μm) of each animal based on cell body recognition using Image Quant 1.43 program (NIH). Immunohistochemistry was performed with antibody against full length AS, before and after proteinase K exposure. Sections were incubated overnight at 4° C. with antibodies against total a-syn (1:500, affinity purified rabbit polyclonal, Millipore) (55), GFAP (mouse monoclonal, Millipore), Iba-1 (mouse monoclonal, Wako laboratories), IL6 (mouse monoclonal Cell Signaling), TLR2 (mouse monoclonal Millipore), TNFα (mouse monoclonal Cell Signaling), IL1beta (mouse monoclonal Abcam) followed by biotin-tagged anti-rabbit or anti-mouse IgG1 (1:100, Vector Laboratories, Inc., Burlingame, Calif.) secondary antibodies, Avidin D-HRP (1:200, ABC Elite, Vector), and visualized with diaminobenzidine. Sections were scanned with a digital Olympus bright field digital microscope (BX41).

Caenorhabditis elegans Studies

Standard conditions were used for C. elegans propagation on NGM (nematode growth medium) plates seeded with E. coli OP50-1 at 20° C. Transgenic (Tg) C. elegans line expressing human AS fused with YFP (AS-YFP) (strain NL5901, Punc-54::AS::YFP) in body wall muscle (50) was obtained from the Caenorhabditis Genetics Center (CGC). The use of these animals allowed for visualization of aggregated AS-YFP in live animals using fluorescence microscopy. Tg AS-YFP nematodes were age-synchronized by hypochlorite bleaching, hatched overnight and were subsequently cultured on NGM plates seeded with E. coli OP50-1 until larval stage L4 (adult day 0). They were then fed for three days solely on fresh NGM plates seeded with either curli-producing E. coli or mutant E. coli unable to produce curli (non-curli mutant). Fluorescence microscopy was performed on immobilized live animals to visualize AS-YFP using an eVOS microscope (Life Technologies) and imaging was acquired under identical conditions for nematodes fed with either wild-type or mutant bacteria. The experiments were repeated three times, each with 10 worms for each treatment group. As characterized previously (50), AS aggregates are recognized as fluorescent foci of inclusions containing AS-YFP within muscle cells in this C. elegans model.

Congo red staining of worms was performed using the procedures modified from that described previously (51). Worms were fixed by incubation in 4% paraformaldehyde in Dulbecco's phosphate buffered saline for 15 h at 4° C., and were permeablized at 37° C. for 15 h in a solution of 1% Triton X-100, 5% beta-mercaptoethanol, 125 mM Tris-HCl, pH 7.5. Worms were then mounted on glass slide and stained with 0.5 mg/ml Congo red in 50% ethanol for 1 min. Destaining was immediately carried out using several rinses of 50% ethanol until solution became colorless, and then one rinse each with 75% ethanol, 50% ethanol, and water. A drop of Fluoromount was applied and stained worms were visualized for red fluorescence using a Leica fluorescence microscope.

### Statistical Analysis

GraphPad Prism (GraphPad Software, San Diego, Calif.) was used for statistical analysis. All data are presented as means ±s.e.m and were analyzed for statistical significance by using either unpaired t test or two-way ANOVA (general linear model) followed by Bonferroni's multiple comparison post-test. Ordinal logistic regression was used to compare the OR for gut AS deposition (49). The statistical software package R was also used. One-way ANOVA post hoc Dunnet p<0.05 was used when comparing to control.

### Cytokine ELISA Assays

Serum samples were collected from 20-21 month-old male Fisher (F344) rats treated with or without curli-producing E. coli, as well as control rats without exposure to bacteria (N=7-8 per group). Following the manufacturer's instruction, serum IL-1β, IL-6, IL-10, INF-γ, and TNF-α were measured by R&D System Quantikine ELISA kits and serum TLR-2 was determined by an ELISA kit from Antibodies-Online (Atlanta Ga.). Data were expressed as optical density (O.D.) value at 450 nm with double wells per sample and the wavelength correction was set at 570 nm. A standard curve was created for each tested cytokine at each time to determine the concentration of the target cytokine concentration in each sample. The concentration of the positive control sample was within the linear range of the standard curve. Results presented as means ±SE, and statistical analysis was done using Prism software. One-way analysis of variance (ANOVA) with Turkey post-hoc test was used for multiple comparisons among groups. p <0.05 was considered statistically significant.

### Cytokine IL-1β, IL-6, IL-10, TNF-α, and INF-γ

Serum INF-γ was detected from all three groups of rats (FIG. 6A). The levels of INF-γ were not significantly different among the three groups. (Treated with curli-producing E. coli group: 31.8±14.2 pg/ml, Treated without curli-producing E. coli group: 15.5±7.8 pg/ml, control group: 55.3±24.1 pg/ml). The serum levels of IL-1β, IL-6, IL-10, and TNF-α were low and could not be detected. See FIG. 6A.

Although particular embodiments of the invention have been illustrated by the foregoing exemplary embodiments, it should be understood that the examples are illustrative only and not intended to be limiting. One of skill in the art will recognize that methods and materials similar or equivalent to those described herein can be used in the practice of the present invention, and numerous changes in the details of implementation of the disclosed subject matter may be made without departing from the spirit and scope of the disclosed subject matter. The scope of the invention is limited only by the claims.

### REFERENCES

1. Walker, L. C. & Jucker, M. Amyloid by default. Nature neuroscience 14, 669-670 (2011).
2. Desplats, P., et al. Inclusion formation and neuronal cell death through neuron-to-neuron transmission of alpha-synuclein. Proceedings of the National Academy of Sciences of the United States of America 106, 13010-13015 (2009).
3. Prusiner, S. B. Cell biology. A unifying role for prions in neurodegenerative diseases. Science 336, 1511-1513 (2012).
4. Friedland, R. P. Mechanisms of molecular mimicry involving the microbiota in neurodegeneration. Journal of Alzheimer's disease : JAD 45, 349-362 (2015).
5. Soto, C. Transmissible proteins: expanding the prion heresy. Cell 149, 968-977 (2012).
6. Braak, H., et al. Staging of brain pathology related to sporadic Parkinson's disease. Neurobiology of aging 24, 197-211 (2003).
7. van Keulen, L. J., Vromans, M. E. & van Zijderveld, F. G. Early and late pathogenesis of natural scrapie infection in sheep. APMIS : acta pathologica, microbiologica, et immunologica Scandinavica 110, 23-32 (2002).
8. Hufnagel, D. A., Tukel, C. & Chapman, M. R. Disease to dirt: the biology of microbial amyloids. PLoS pathogens 9, e1003740 (2013).
9. Larsen, P., et al. Amyloid adhesins are abundant in natural biofilms. Environ Microbiol 9, 3077-3090 (2007).
10. Cherny, I., et al. The formation of Escherichia coli curli amyloid fibrils is mediated by prion-like peptide repeats. Journal of molecular biology 352, 245-252 (2005).
11. Evans, M. L., et al. The bacterial curli system possesses a potent and selective inhibitor of amyloid formation. Mol Cell 57, 445-455 (2015).
12. Lundmark, K., Westermark, G. T., Olsen, A. & Westermark, P. Protein fibrils in nature can enhance amyloid protein A amyloidosis in mice: Cross-seeding as a disease mechanism. Proceedings of the National Academy of Sciences of the United States of America 102, 6098-6102 (2005).
13. Solomon, A., et al. Amyloidogenic potential of foie gras. Proceedings of the National Academy of Sciences of the United States of America 104, 10998-11001 (2007).
14. Colonna, M. & Wang, Y. TREM2 variants: new keys to decipher Alzheimer disease pathogenesis. Nat Rev Neurosci 17, 201-207 (2016).
15. Zenaro, E., et al. Neutrophils promote Alzheimer's disease-like pathology and cognitive decline via LFA-1 integrin. Nature medicine 21, 880-886 (2015).
16. Holmes, C. Review: systemic inflammation and Alzheimer's disease. Neuropathology and applied neurobiology 39, 51-68 (2013).
17. Akiyama, H., et al. Inflammation and Alzheimer's disease. Neurobiology of aging 21, 383-421 (2000).
18. Lathrop, S. K., et al. Peripheral education of the immune system by colonic commensal microbiota. Nature 478, 250-254 (2011).
19. Tukel, C., et al. Toll-like receptors 1 and 2 cooperatively mediate immune responses to curli, a common amyloid from enterobacterial biofilms. Cellular microbiology 12, 1495-1505 (2010).
20. Kim, C., et al. Antagonizing Neuronal Toll-like Receptor 2 Prevents Synucleinopathy by Activating Autophagy. Cell Rep 13, 771-782 (2015).
21. Phillips, R. J., Walter, G. C., Ringer, B. E., Higgs, K. M. & Powley, T. L. Alpha-synuclein immunopositive aggregates in the myenteric plexus of the aging Fischer 344 rat. Experimental neurology 220, 109-119 (2009).
22. Baker, P. J., Dixon, M. & Roopenian, D. C. Genetic control of susceptibility to Porphyromonas gingivalis-induced alveolar bone loss in mice. Infection and immunity 68, 5864-5868 (2000).
23. van Ham, T. J., et al. C. elegans model identifies genetic modifiers of alpha-synuclein inclusion formation during aging. PLoS genetics 4, e1000027 (2008).
24. Hartman, K., et al. Bacterial curli protein promotes the conversion of PAP248-286 into the amyloid SEVI: cross-seeding of dissimilar amyloid sequences. PeerJ 1, e5 (2013).
25. Guo, J. L. & Lee, V. M. Seeding of normal Tau by pathological Tau conformers drives pathogenesis of Alzheimer-like tangles. The Journal of biological chemistry 286, 15317-15331 (2011).
26. Klingelhoefer, L. & Reichmann, H. Pathogenesis of Parkinson disease—the gut-brain axis and environmental factors. Nature reviews. Neurology 11, 625-636 (2015).
27. Nussbaum-Krammer, C. I. & Morimoto, R. I. Caenorhabditis elegans as a model system for studying non-cell-autonomous mechanisms in protein-misfolding diseases. Dis Model Mech 7, 31-39 (2014).
28. Allen Reish, H. E. & Standaert, D. G. Role of alpha-synuclein in inducing innate and adaptive immunity in Parkinson disease. J Parkinsons Dis 5, 1-19 (2015).
29. Olmos-Alonso, A., et al. Pharmacological targeting of CSF1R inhibits microglial proliferation and prevents the progression of Alzheimer's-like pathology. Brain : a journal of neurology 139, 891-907 (2016).
30. Reed-Geaghan, E. G., Savage, J. C., Hise, A. G. & Landreth, G. E. CD14 and toll-like receptors 2 and 4 are required for fibrillar A {beta}-stimulated microglial activation. The Journal of neuroscience : the official journal of the Society for Neuroscience 29, 11982-11992 (2009).
31. Milatovic, D., Zaja-Milatovic, S., Montine, K. S., Shie, F. S. & Montine, T. J. Neuronal oxidative damage and dendritic degeneration following activation of CD14-dependent innate immune response in vivo. Journal of neuroinflammation 1, 20 (2004).
32. Gallo, P. M., et al. Amyloid-DNA Composites of Bacterial Biofilms Stimulate Autoimmunity. Immunity 42, 1171-1184 (2015).
33. Selkoe, D. J. & Hardy, J. The amyloid hypothesis of Alzheimer's disease at 25 years. EMBO Mol Med (2016).
34. Poehler, A. M., et al. Autophagy modulates SNCA/alpha-synuclein release, thereby generating a hostile microenvironment. Autophagy 10, 2171-2192 (2014).
35. Chorell, E., et al. Bacterial Chaperones CsgE and CsgC Differentially Modulate Human alpha-Synuclein Amyloid Formation via Transient Contacts. PloS one 10, e0140194 (2015).
36. De Strooper, B. & Karran, E. The Cellular Phase of Alzheimer's Disease. Cell 164, 603-615 (2016).
37. Condello, C., Yuan, P., Schain, A. & Grutzendler, J. Microglia constitute a barrier that prevents neurotoxic protofibrillar Abeta42 hotspots around plaques. Nature communications 6, 6176 (2015).
38. Chen, A., et al. Multiplex analyte assays to characterize different dementias: brain inflammatory cytokines in poststroke and other dementias. Neurobiology of aging 38, 56-67 (2016).
39. Raby, A. C., et al. Soluble TLR2 reduces inflammation without compromising bacterial clearance by disrupting TLR2 triggering. J Immunol 183, 506-517 (2009).
40. Oppong, G. O., et al. Biofilm-associated bacterial amyloids dampen inflammation in the gut: oral treatment with curli fibres reduces the severity of hapten-induced colitis in mice. NPJ Biofilms Microbiomes 1(2015).
41. Gilchrist, K. B., Garcia, M. C., Sobonya, R., Lipke, P. N. & Klotz, S. A. New features of invasive candidiasis in humans: amyloid formation by fungi and deposition of serum amyloid P component by the host. J Infect Dis 206, 1473-1478 (2012).
42. Schwartz, K. & Boles, B. R. Microbial amyloids—functions and interactions within the host. Current opinion in microbiology 16, 93-99 (2013).
43. Aguzzi, A. Neurodegeneration: Alzheimer's disease under strain. Nature 512, 32-34 (2014).
44. Peelaerts, W., et al. alpha-Synuclein strains cause distinct synucleinopathies after local and systemic administration. Nature 522, 340-344 (2015).
45. Wilmes, P., Heintz-Buschart, A. & Bond, P. L. A decade of metaproteomics: where we stand and what the future holds. Proteomics 15, 3409-3417 (2015).
46. Phillips, R. J., Walter, G. C., Ringer, B. E., Higgs, K. M. & Powley, T. L. Alpha-synuclein immunopositive aggregates in the myenteric plexus of the aging Fischer 344 rat. Experimental neurology 220, 109-119 (2009).
47. Baker, P. J., DuFour, L., Dixon, M. & Roopenian, D. C. Adhesion molecule deficiencies increase Porphyromonas gingivalis-induced alveolar bone loss in mice. Infection and immunity 68, 3103-3107 (2000).
48. Hsu, L. J., Jan, M. S. & Lin, Y. S. In vivo staphylococcal enterotoxin B (SEB)-primed murine splenocytes secrete mediators which suppress CD25(hi) expression and cell cycle progression of naive splenocytes in response to SEB in vitro. Cell Immunol 201, 50-57 (2000).
49. McCullagh, P. Regression Models for Ordinal Data. J. R. Statist. Soc. 42, 109-142 (1980).
50. van Ham, T. J., et al. C. elegans model identifies genetic modifiers of alpha-synuclein inclusion formation during aging. PLoS genetics 4, e1000027 (2008).
51. Wu, Y., et al. Amyloid-beta-induced pathological behaviors are suppressed by Ginkgo biloba extract EGb 761 and ginkgolides in transgenic Caenorhabditis elegans. The Journal of neuroscience : the official journal of the Society for Neuroscience 26, 13102-13113 (2006).
52. Rapsinski G J, et al, Toll-like receptor 2 and NLRP3 cooperate to recognize a functional bacterial amyloid, curli. Infect Immun; 83:693-701 (2015).
53. Minter M R, et al, Antibiotic-induced perturbations in gut microbial diversity influences neuro-inflammation and amyloidosis in a murine model of Alzheimer's disease. Sci Rep. 21; 6:30028 (2016).
54. Harach T, et al, Reduction of Alzheimer's disease beta-amyloid pathology in the absence of gut microbiota arXiv:1509.02273[q-bio.MN]
55. Hsu L J, et al, Alpha-synuclein promotes mitochondrial deficit and oxidative stress. Am J Pathol. 2000;157:401-10 (2000).
56. Phillips R J, et al, Alpha-synuclein expression patterns in the colonic submucosal plexus of the aging Fischer 344 rat: implications for biopsies in aging and neurodegenerative disorders? Neurogastroenterol Motil 25:e621-33 (2013).
57. Daep C A, et al, Structural dissection and in vivo effectiveness of a peptide inhibitor of Porphyromonas gingivalis adherence to Streptococcus gordonii. Infect Immun. 2011 Jan;79(1):67-74 (2011).
58. Epstein E A, et al, Spatial clustering of the curlin secretion lipoprotein requires curli fiber assembly. J Bacteriol. 2009 Jan;191(2):608-15 (2009).

1. A method of preventing, inhibiting and/or decreasing protein deposits of a human amyloid protein and/or neuroinflammation in the central nervous system by reducing an amount of exposure to a pathogenic bacterial amyloid in the gastrointestinal tract.

2. A method of preventing, inhibiting and/or treating a proteinopathy by reducing an amount of exposure to a pathogenic bacterial amyloid in the gastrointestinal tract.

3. The method of claim 1 wherein the human amyloid protein is selected from the group consisting of alpha-synuclein (AS), amyloid beta (AB), Tau, FUS or TDP43.

4. The method of claim 1 wherein the pathogenic bacterial amyloid is an amyloid produced by a bacterium of a phylum selected from the group consisting of Bacteroidetes, Proteobacteria, Firmicutes and Thermodesulfobacteria.

5. The method of claim 1 wherein the pathogenic bacterial amyloid is the E. coli curli protein or a Bacteroidetes, Proteobacteria, Firmicutes or Thermodesulfobacteria homologue of the E. coli curli protein.

6. The method of claim 1 wherein the reduction in exposure is accomplished by administering at least one antibiotic to reduce the relative abundance of the bacterial strain producing the pathogenic bacterial amyloid.

7. The method of claim 1 wherein the reduction in exposure is accomplished by administering at least one prebiotic to increase the relative abundance of non-pathogenic bacterial strains which will at least partially displace the bacterial strain producing the pathogenic bacterial amyloid.

8. The method of claim 1 wherein the reduction in exposure is accomplished by administering at least one prebiotic to reduce the relative abundance of the bacterial strain producing the pathogenic bacterial amyloid relative to a non-pathogenic bacterial strain which will at least partially displace the pathogenic strain.

9. The method of claim 1 wherein the reduction in exposure is accomplished by administering at least one probiotic to provide at least one non-pathogenic bacterial strain which will at least partially displace the bacterial strain producing the pathogenic bacterial amyloid.

10. The method of claim 1 wherein the reduction in exposure is accomplished by administering a pharmaceutical preparation with an active agent that alters the pathogenic bacterial amyloid to a nonpathogenic form.

11. The method of claim wherein the reduction in exposure is accomplished by administering a pharmaceutical preparation with an active agent that alters the interaction between the pathogenic bacterial amyloid and a human amyloidogenic protein such that nucleation of the aggregated form of the human amyloidogenic protein is inhibited, prevented and/or reversed.

12. The method of claim 1 wherein the reduction in exposure is accomplished by administering a pharmaceutical preparation with an active agent that alters the interaction between the pathogenic bacterial amyloid and cells of the human gastrointestinal tract such that nucleation of the aggregated form of a human amyloidogenic protein is inhibited, prevented and/or reversed.

13. The method of claim 12 wherein the interaction is uptake of the pathogenic bacterial amyloid into cells of the human gastrointestinal tract.

14. The method of claim 1 wherein the reduction in exposure is accomplished by administering a pharmaceutical preparation with an active agent which immunizes a subject against the pathogenic bacterial amyloid such that the subject's immune system reduces the relative abundance of the pathogenic bacteria.

15. The method of claim 1 wherein the reduction in exposure is accomplished by orally administering a pharmaceutical preparation with an active agent which decreases production of bacterial amyloid.

16. The method of claim 2 wherein the human amyloid protein is selected from the group consisting of alpha-synuclein (AS), amyloid beta (AB), Tau, FUS or TDP43.

17. The method of claim 2 wherein the pathogenic bacterial amyloid is an amyloid produced by a bacterium of a phylum selected from the group consisting of Bacteroidetes, Proteobacteria, Firmicutes and Thermodesulfobacteria.

18. The method of claim 2 wherein the pathogenic bacterial amyloid is the E. coli curli protein or a Bacteroidetes, Proteobacteria, Firmicutes or Thermodesulfobacteria homologue of the E. coli curli protein.

19. The method of claim 2 wherein the reduction in exposure is accomplished by administering at least one antibiotic to reduce the relative abundance of the bacterial strain producing the pathogenic bacterial amyloid.

20. The method of claim 2 wherein the reduction in exposure is accomplished by administering at least one prebiotic to increase the relative abundance of non-pathogenic bacterial strains which will at least partially displace the bacterial strain producing the pathogenic bacterial amyloid.

21. The method of claim 2 wherein the reduction in exposure is accomplished by administering at least one prebiotic to reduce the relative abundance of the bacterial strain producing the pathogenic bacterial amyloid relative to a non-pathogenic bacterial strain which will at least partially displace the pathogenic strain.

22. The method of claim 2 wherein the reduction in exposure is accomplished by administering at least one probiotic to provide at least one non-pathogenic bacterial strain which will at least partially displace the bacterial strain producing the pathogenic bacterial amyloid.

23. The method of claim 2 wherein the reduction in exposure is accomplished by administering a pharmaceutical preparation with an active agent that alters the pathogenic bacterial amyloid to a nonpathogenic form.

24. The method of claim 2 wherein the reduction in exposure is accomplished by administering a pharmaceutical preparation with an active agent that alters the interaction between the pathogenic bacterial amyloid and a human amyloidogenic protein such that nucleation of the aggregated form of the human amyloidogenic protein is inhibited, prevented and/or reversed.

25. The method of claim 2 wherein the reduction in exposure is accomplished by administering a pharmaceutical preparation with an active agent that alters the interaction between the pathogenic bacterial amyloid and cells of the human gastrointestinal tract such that nucleation of the aggregated form of a human amyloidogenic protein is inhibited, prevented and/or reversed.

26. The method of claim 25 wherein the interaction is uptake of the pathogenic bacterial amyloid into cells of the human gastrointestinal tract.

27. The method of claim 2 wherein the reduction in exposure is accomplished by administering a pharmaceutical preparation with an active agent which immunizes a subject against the pathogenic bacterial amyloid such that the subject's immune system reduces the relative abundance of the pathogenic bacteria.

28. The method of claim 2 wherein the reduction in exposure is accomplished by orally administering a pharmaceutical preparation with an active agent which decreases production of bacterial amyloid.
